(19) United States
(12) Patent Application Publication
(10) Pub. No.: US 2005/0153003 A1
(43) Pub. Date: Jul. 14, 2005

(54) Anti-aging cosmetic composition and method of application

(75) Inventors: Gopinathan K. Menon, Wayne, NJ (US); Isabella L. Morelli-Abrams, Stony point, NY (US)

Correspondence Address: CHARLES N.J. RUGGIERO, ESQ.;OHLANDT, GREELEY, RUGGIERO & PERLE, L.L.P., 10th FLOOR, ONE LANDMARK SQUARE, STAMFORD, CT, 06901-2682, US

(73) Assignee: Avon Products, Inc.

(21) Appl. No.: 11/078,030

(22) Filed: Mar. 11, 2005

Related U.S. Application Data
(63) Continuation of application No. 09/723,459, filed on Nov. 28, 2000, now abandoned.

(51) Int. Cl. A61K 35/78

(52) U.S. Cl. 424/769

(57) ABSTRACT

The present invention provides topical compositions and methods to prevent and/or ameliorate the effects of aging on skin and/or hair. The present invention also provides topical compositions and methods to improve the aesthetic appearance of skin, scalp and/or hair. The invention utilizes the topical application of crape myrtle extract to skin, hair and/or scalp.

### BACKGROUND OF THE INVENTION

1. Field of the Invention

The present invention relates to a composition that improves the aesthetic appearance of skin and hair. More particularly, the present invention relates to a topical composition comprising crape myrtle extract to treat aging skin.

2. Description of the Prior Art

The appearance and physical properties of human skin and hair cells (hereinafter collectively referred to as “skin cells”), especially keratinocytes, fibroblasts and sebocytes, change with age. In particular, the ability of these cells to transport in and utilize glucose decreases.

A loss in take up and utilization of glucose manifests in an increased concentration of glucose in the extracellular matrix and a decrease in cell metabolism. Decreased cell metabolism reduces both cell replication and cell vitality. For example, a reduction in the rate of replication of basal epidermal cells reduces the thickness of the epidermis and, thus, the skin. In addition, increased extracellular glucose accumulation in the skin leads to what is known as the formation of Advanced Glycation End (AGE) Products and the loss of functional properties of collagen and elastin. Thus, sagging and wrinkling of the skin occurs.

### SUMMARY OF THE INVENTION

It is an object of the present invention to provide a topical composition and method of use to improve the aesthetic appearance of skin and hair.

It is also an object of the present invention to provide a topical composition and method of use to prevent and/or ameliorate the effects of extrinsic and/or intrinsic aging on the skin and the hair.

It is another object of the present invention to provide a topical composition and method of use to prevent and/or reduce wrinkling of the skin.

It is still another object of the present invention to provide a topical composition and method of use to prevent and/or reduce sagging of the skin.

These and other objects and advantages of the present invention are achieved by a topical composition that has an effective amount of crape myrtle extract and a cosmetically acceptable vehicle.

### DETAILED DESCRIPTION OF THE INVENTION

In the present invention, it has been unexpectedly and surprisingly found that a topical composition can be formulated to improve the aesthetic appearance of skin (including lips)and hair, particularly to prevent and/or ameliorate the effects of extrinsic and/or intrinsic aging of the skin. Intrinsic aging may include chronological aging. Extrinsic aging may include photoaging and the effects of environmental pollution.

The present invention comprises crape myrtle extract in a cosmetic vehicle for topical application to skin, scalp and/or hair for a period of time and in an amount sufficient to improve the skin and/or hair condition.

Crape myrtle extract is obtained from the leaves of the crape myrtle plant (lagerstroemia speciosa), sometimes also referred to as the banaba tree. Crape myrtle extract is typically obtained either in powdered form or in liquid form (including a diluent). Crape myrtle extract may also be cell cultured and provided in a cultured cell broth to minimize lot-to-lot, season-to-season and location-to-location variation associated with plant-sourced activities.

Crape myrtle extract contains colosic acid or 2α-hydroxyursolic acid as one of its constituents. The chemical structure for colosic acid is seen in Table III of Murakami et al., Chem. Pharm. Bull. 41 [12] 2129-2131 (1993), which is incorporated herein by reference in its entirety. Other teachings regarding crape myrtle and lagerstroemia speciosa are seen in Kakuda et al., Biosci. Biotech. Biochem. 60 (2) 204-208 (1996) and Kajimoto and Murakami, Nippon Elyo, Shokuryo Gakkaishi 52: 209-218 (1999), both of which are incorporated herein by reference in their entirety.

The effective amount of crape myrtle extract and the duration of its application will vary with the particular condition being treated, the age and physical condition of the person, the severity of the condition, the nature of concurrent therapy, the particular topical vehicle utilized, and like factors in the knowledge and expertise of those skilled in the art. The duration of application may be once or twice a day for a period of one or two weeks or more.

Topical compositions of the present invention comprise crape myrtle extract from about 0.0001 to about 15 weight percent (wt %) based on the total weight of the composition. Preferably, crape myrtle extract is present from about 0.001 to about 10 wt %, and most preferably, present from about 0.001 wt % to about 5 wt % based on the weight of the total composition. The above amounts are based upon “active amount” of crape myrtle extract. The term “active amount” refers to the amount of crape myrtle extract absent diluent, solvent, or any other ingredient added for bulk. In addition, all weight percentages disclosed herein are based upon the total weight of the composition unless otherwise indicated.

The present invention preferably includes a cosmetic vehicle. Such vehicles may take the form of any known in the art suitable for application to skin, hair, or scalp. These vehicles may include water; vegetable oils; mineral oils; esters such as octyl palmitate, isopropyl myristate and isopropyl palmitate; ethers such as dicapryl ether and dimethyl isosorbide; alcohols such as ethanol and isopropanol; fatty alcohols such as cetyl alcohol, cetearyl alcohol, stearyl alcohol and behenyl alcohol; isoparaffins such as isooctane, isododecane and isohexadecane; silicone oils such as cyclomethicone, dimethicone, dimethicone cross-polymer, polysiloxanes and their derivatives preferably organomodified derivatives; hydrocarbon oils such as mineral oil, petrolatum, isoeicosane and polyisobutene; polyols such as propylene glycol, glycerin, butylene glycol, pentylene glycol and hexylene glycol; waxes such as beeswax and botanical waxes; and mixtures of the foregoing. Additional ingredients that may be included in the vehicle are disclosed in U.S. Pat. No. 5,162,378 (column 4, et seq.), which is incorporated herein by reference.

The present composition can be made into any suitable product form, such as an aerosol, cake, cream, ointment, emulsion, essence, gel, lotion, paste, patch, pencil, serum, towelette, mask, spray and stick. If desired, the composition can be formulated into make-up cosmetics, shampoos and conditioners.

The present composition may further comprise one or more of the following additional ingredients: anesthetics, anti-allergenics, antimicrobials, antifungals, anti-inflammatories, antiseptics, chelating agents, colorants, depigmenting agents, emollients, exfollients, fragrances, emulsifiers, humectants, insect repellents, lubricants, moisturizers, pharmaceutical agents, preservatives, skin penetration enhancers, skin protectants, stabilizers, sunscreens, surfactants, thickeners, viscosity modifiers, and vitamins. A preferred composition comprises the combination of crape myrtle extract with any one or more of vitamin A, vitamin C and/or bioflavonoids.

The compositions of the present invention may be applied topically to the skin, scalp, and hair to reduce the extrinsic and/or intrinsic aging on and to enhance to the aesthetic appearance of the skin, scalp and/or hair.

Topically applying compositions of the present invention to the skin can enhance and improve the aesthetic appearance of skin by, among other improvements, decreasing skin fragility; preventing and reversing deterioration of collagen and/or elastin; preventing skin atrophy; promoting/accelerating cell turnover; improving skin firmness/plumpness; improving skin texture; decreasing fine lines and wrinkles; improving skin tone; enhancing skin thickness; restoring skin luster; minimizing signs of fatigue; reducing skin dryness; and enhancing overall skin health. The benefits of increased cell proliferation and, thus, increased plumpness of the skin, can also decrease the extent and/or duration of bruising visible after physical trauma.

Topically applying compositions of the present invention to the scalp and/or hair can enhance and improve the aesthetic appearance of hair by, among other improvements, increasing the amount and/or rate of hair growth; increasing hair thickness and/or length, improving hair luster and shine, improving hair manageability, and improving overall hair health.

### EXAMPLE 1

Two assays employing crape myrtle extract were tested for effect in stimulating the formation of keratinocytes. The compositions varied in concentration of crape myrtle extract. A test solution having 1 wt % to 2 wt % of crape myrtle extract in a polar solvent mixture was added to cell culture media to form the assay compositions. Two assay compositions were tested having test solution concentrations of 0.001 wt % and 1.0 wt %, based on the total weight of the assay compositions, by the addition to a culture having keratinocytes. A control assay was prepared in substantially the same manner as the test assays except that the crape myrtle was deleted.

The assays employing test solution (containing crape myrtle) concentrations of 0.001 wt % and 1.0 wt % exhibited a multiple of 1.6 times increase in keratinocyte production compared to the control.

### EXAMPLE 2

Two assays employing crape myrtle extract were tested for effect in stimulating the formation of fibroblasts. The compositions varied in concentration of crape myrtle extract. The test solution employed was the same as in Example 1. Two assay compositions containing test solution concentrations of 0.001 wt % and 7.0 wt %, based on the total weight of the assay, were tested by addition to a culture having fibroblasts. A control assay was prepared in substantially the same manner as the test assays except that the crape myrtle was deleted.

The assays employing test solution (containing crape myrtle) concentrations of 0.001 wt % and 7.0 wt % exhibited a multiple of 1.24 times increase in fibroblast production compared to the control.

It should be understood that the foregoing description is only illustrative of the present invention. Various alternatives and modifications can be devised by those skilled in the art without departing from the invention. Accordingly, the present invention is intended to embrace all such alternatives, modifications and variances that fall within the scope of the appended claims.

1. A topical composition comprising:
crape myrtle extract in an amount effective to improve the aesthetic appearance of skin, scalp and/or hair, and
a cosmetically acceptable vehicle.

2. The composition of claim 1, wherein the crape myrtle extract is present in from about 0.0001 to about 15 wt % based on the total weight of the composition.

3. The composition of claim 1, wherein the crape myrtle extract is present from about 0.001 wt % to about 10 wt % based on the total weight of the composition.

4. The composition of claim 1, wherein the crape myrtle extract is obtained from the leaf cells of lagerstroemia speciosa.

5. The composition of claim 1, wherein the composition is in a product form selected from the group consisting of aerosol, cake, cream, ointment, emulsion, essence, gel, lotion, shampoos, conditioners, make-up, towelette, mask, paste, patch, pencil, serum, spray and stick.

6. The composition of claim 1, further comprising one or more additional ingredients selected from the group consisting of anesthetics, anti-allergenics, antimicrobials, antifungals, anti-inflammatories, antiseptics, chelating agents, colorants, depigmenting agents, emollients, emulsifiers, exfollients, fragrances, humectants, insect repellents, lubricants, moisturizers, pharmaceutical agents, preservatives, skin penetration enhancers, skin protectants, stabilizers, sunscreens, surfactants, thickeners, viscosity modifiers, and vitamins.

7. The composition of claim 1, further comprising one or more sunscreens.

8. The composition of claim 1, wherein the crape myrtle extract includes colosic acid.

9. The composition of claim 1, further comprising at least one of vitamin A, vitamin C, and bioflavonoids.

10. A method of improving the aesthetic appearance of skin, comprising topically applying to the skin:
a composition comprising an effective amount of crape myrtle extract; and
a cosmetically acceptable vehicle.

11. The method of claim 10, wherein the crape myrtle extract is applied in an amount from about 0.0001 wt % to about 15 wt % based on the total weight of the composition.

12. The method of claim 10, wherein the crape myrtle extract is applied in an amount from about 0.001 wt % to about 10 wt % based on the total weight of the composition.

13. The method of claim 10, wherein the crape myrtle extract is applied to the skin at least once daily for a period of time sufficient to improve the aesthetic appearance of skin.

14. The method of claim 10, wherein the improvement in aesthetic appearance is selected from the group consisting of:
a) decreasing skin fragility;
b) preventing and reversing deterioration of collagen and/or elastin;
c) preventing skin atrophy;
d) promoting/accelerating cell turnover;
e) improving skin firmness/plumpness;
f) improving skin texture;
g) decreasing fine lines and/or wrinkles;
h) improving skin tone;
i) enhancing skin thickness;
j) restoring skin luster;
k) minimizing signs of fatigue; and
l) reducing skin dryness.

15. A method of preventing and/or ameliorating the effects of extrinsic and/or intrinsic aging on skin comprising topically applying to the skin a composition comprising an effective amount of crape myrtle extract and a cosmetically acceptable vehicle.

16. The method of claim 15, wherein the effects of skin aging are selected from the group consisting of:
a) fragile skin;
b) sagging skin;
c) fine lines and/or wrinkles;
d) thinning skin;
e) lack-luster skin;
f) fatigued skin; and
g) dry skin.

17. The method of claim 15, wherein the crape myrtle extract is applied to the skin at least once daily for a period of time sufficient to prevent and/or ameliorate the effects of skin aging.

18. The method of claim 15, wherein the intrinsic aging comprises chronological aging.

19. The method of claim 15, wherein the extrinsic aging comprises photoaging and/or the effects of pollution.

20. The method of claim 15, wherein the crape myrtle extract is applied to the skin in an amount from about 0.0001 wt % to about 15 wt % of the total composition.

21. The method of claim 15, wherein the crape myrtle extract is applied in an amount from about 0.001 wt % to about 10 wt % based on the total weight of the composition.

22. A method of improving the aesthetic appearance of hair comprising applying to hair and/or scalp crape myrtle extract in an amount effective to improve the aesthetic appearance of hair.

23. The method of claim 22, wherein the improvement in aesthetic appearance of hair includes at least one of the following:
a) increasing hair thickness;
b) increasing rate of hair growth;
c) improving hair luster and shine;
d) improving manageability; and
e) overall hair health.

24. The method of claim 22, wherein the crape myrtle extract is applied to the hair and/or scalp in a topical composition comprising a vehicle and from about 0.0001 wt % to about 15 wt % of crape myrtle extract.

25. The method of claim 22, wherein the crape myrtle extract is applied to the hair and/or scalp at least once per day for a period of time sufficient to improve the aesthetic appearance of the hair.
